(19) United States
(12) Patent Application Publication
(10) Pub. No.: US 2021/0401022 A1
(43) Pub. Date: Dec. 30, 2021

(54) NON-SOY, LEGUME, PROTEIN MATERIAL AND METHOD OF MAKING SUCH

(71) Applicant: Puris Proteins, LLC, Minneapolis, MN (US)

(72) Inventors: KUSHAL NARAYAN CHANDAK, St. Louis Park, MN (US); ALEXANDER EDWARD KING, Apple Valley, MN (US); MARK ROBERT POWERS, Plymouth, MN (US)

(21) Appl. No.: 16/911,322

(22) Filed: Jun. 24, 2020

(51) Int. Cl. A23L 33/185; A23J 1/14; A23J 3/34; A23J 3/14; A23C 11/06; A23L 2/66; A23C 20/02; A23C 9/13; A23L 2/39; A61K 47/46

(52) U.S. Cl. CPC A23L 33/185; A23J 1/148; A23J 3/347; A23J 3/14; A23C 11/06; A23V 2002/00; A23C 20/025; A23C 9/1315; A23L 2/39; A61K 47/46; A23L 2/66

(57) ABSTRACT

The present disclosure relates to a non-soy, legume, protein material that is at least 50% dry weight non-soy, legume, protein; has a pH of about 4-8; and has a Nitrogen Solubility Index of greater than 40%. Preferably, the non-soy, legume, protein material of this disclosure additionally has a Protein Dispersability Index of greater than about 70%. Preferably, the non-soy, legume, protein material comprises at least 20% dry weight pea protein, meets USDA Organic Certification requirements, and is not genetically modified.

### BACKGROUND OF THE DISCLOSURE

The present disclosure is broadly concerned with non-soy, legume, protein material that can be used to make nutritious, good tasting, high protein content food products without using allergen protein sources (e.g., soy, milk, gluten). In particular, but not exclusively, the present disclosure is concerned with a pea protein material that can be used in high quantities in food products, which are finished food products or intermediate food products.

The present disclosure comprises methods for making this non-soy, legume, protein material that involves solubilizing and separating plant protein matter from ground, de-hulled plant matter (such as from peas, lentils, fava beans, lupin or broad beans); treating the protein matter; and then precipitating the protein matter in a form that has unique functional characteristics that are useful in food products. Optionally, non-soy, legume, protein matter can be precipitated and then treated. The resultant non-soy, legume, protein material can then be used to make food products (for animals or humans) with a smooth, creamy mouthfeel and a product viscosity that has acceptable viscosity, such as a pourable viscosity. Being pourable creates acceptable intermediate food product character so as to allow for movement of product through pipes, pumps, tanks, and filler heads during food product processing. Being pourable allows consumer desired finished product viscosity (i.e., thickness), such as a smooth, flowing texture of a RTD beverage when it is in a bottle or glass, even when the beverage formula has s high protein content (e.g., 20% protein material). With most current plant protein products, addition of high amounts of protein to a food product creates a gritty textured finished product due to not-solubilized, dispersed, or dissolved protein. With some current plant protein products, addition level of protein is limited because the protein absorbs so much water that the protein suspension or food product is too viscous to process and/or consume.

The resultant non-soy, legume, protein material of the presently disclosed process can also be used to make supplements, pharmaceuticals, and industrial products. All mentions of the disclosed non-soy, legume, protein material towards use in food products, also covers similar use in supplements, pharmaceuticals and industrial products.

In particular, but not exclusively, the present disclosure is concerned with a non-soy, legume, protein material, which at low and high concentration levels in food products, solves the current problem of manageable product viscosity and product texture (e.g., mouthfeel).

Product formulators have several potential sources of protein material that they could use to perform these protein functions in food products. However, not all protein materials function the same way, whether that is because of their source or because of their chemical content, physical structure, and/or composition. With many currently marketed plant protein materials, addition of high concentrations of that protein material to food product formulas creates a gritty textured finished product due to non-solubilized, non-dispersed, and/or non-dissolved protein material. With many available plant based protein materials, addition level of protein material is limited because the protein material absorbs so much water that the food product (in intermediate or finished form) is too viscous to process and/or drink. Particle size and physical structure of a protein material can also affect food product texture. For example, the tongue can feel a three-dimensional particle as “grit”, if the particles are too large. If the particles are small enough, the tongue will not feel them. If the particles are flat, like platelets, then the tongue will not feel them or will feel them as “slippery” or “smooth”, especially if the material the particles are in is viscous.

Some of the protein sources that are currently available to product formulators comprise wheat (e.g., gluten), animal (e.g., egg albumin, milk casein, milk whey), and soybeans. One challenge to product formulators is that these protein sources can be perceived to have disease or allergen negative physical effects for many consumers. For example, soybeans, wheat gluten, and milk sourced proteins are allergens that FDA requires to be specifically identified on food product labels. Others, such as wheat and milk based proteins, are associated with physical intolerance, either directly (e.g., gluten in wheat sources) or through associated ingredients (e.g., lactose in milk sources). Consumers for ethical or sustainability reasons avoid some of these protein sources (e.g., animal sources).

Protein material also affects food product flavor, aroma, and color. Some protein materials have unique flavors and aromas associated with them, such as the beany, earthy, and/or musty flavor associated with soybean protein material. Milk based proteins often have burnt and/or cooked milk flavors associated with them. Usually, the most bland flavors and aromas are the most preferred by product developers as those protein materials create a bland platform upon which to build unique food product flavors. The color supplied by a protein material is often affected by the presence of non-protein components in the protein material, such as legume hull fiber. Processing of the protein material can affect color through caramelization of lactose content in milk based proteins, and through Maillard browning in all protein sources. As with flavor and aroma, product developers prefer the blandest, whitest platform upon which to build unique food product colors. If the desired food product color was dark brown, then most protein sources would be good sources for the nitrogen and carbohydrate required for Maillard Browning.

Research and product development has been done by many commercial interests to create finished consumer products with soybean based proteins used as replacements for wheat, milk, and/or animal based proteins for many of the already stated reasons. However, FDA considers soybean proteins as allergenic ingredients, and so they must be listed on labels. Many consumers do not like the musty, beany flavor or the flatulence effect unique to soybean protein materials.

The role (i.e., function) of protein material in consumer food products varies with each type of finished food product, supplement, pharmaceutical, or industrial material. The role is dependent on what consumers want the finished product for. Consumers want high protein content in their food products, especially in those food products consumed as replacements (or alternatives) for meat, eggs, milk, or soybean based proteins. Consumers attempting to control their weight also want the satiety benefits of high protein content. Consumers who are athletes want food products with high protein content for muscle recovery and growth. However, there is a limit on how much protein a formulator can be add to food product formula. Protein solubility is critical to developing food products with high protein content. The resulting product texture and flavor are critical to consumer acceptance of the high protein product.

An example of a food product form often chosen by consumers to meet these protein wants and needs are beverages. The beverages can be plant based milks, Ready-To-Drink (RTD), and dry based beverages (DBB). Unfortunately, product developers have found that some protein materials have limited water solubility, which is the cause of the proteins functionality. In beverages, insoluble or non-dispersed protein can make beverage food products intolerability gritty in texture. Some protein materials have too much water absorption ability, which can make beverages become too thick to process and to consume.

The ability of a protein to interact with water creates protein solubility, which is key to the functionality of that protein. For example, dispersability, solubility, suspension, sedimentation stability (i.e., precipitation, suspension), viscosity building, emulsification, creaminess building, and body building are all functions desired from proteins in food products and all such functions are based on protein's solubility in water. The functionality of plant based protein materials can be affected by the physical nature of the protein, such as its size, physical configuration, and charged nature. Some of the physical nature of a protein material can be modified by the way the protein material has been processed or by the environment the protein material finds itself while in a food product (e.g., presence of food grade buffers, protein linking agents, solutes, acids, base, enzymes, heat, and/or sheer).

A continuing challenge to plant protein material suppliers is creating protein material that has not only the physical functionality desired, but also the flavor and color desired. Unless a plant based protein material is bland in flavor, aroma, and color, the organoleptic properties of the protein material could predominate or overwhelm the flavor or color ingredients added to a food product formulation. And as more protein material is added to a formulation, the organoleptic properties of that protein material will become more problematic. For example, protein material sourced from soybeans can have a beany, musty flavor that could be difficult to flavor formulate around.

Therefore, there is a need for a non-soy, legume, protein material with the functionality and organoleptic properties that product developers could use to meet the various protein functions required to create finished products with the physical and organoleptic characteristics desired by consumers. These finished products comprise, but are not limited to, human food, animal food, supplements, pharmaceutical, and industrial products.

### SUMMARY OF DISCLOSURE

The present disclosure relates to a non-soy, legume, protein material that is at least 50% dry weight non-soy, legume, protein; has a pH of about 4-8; and has a Nitrogen Solubility Index of greater than 40%. Preferably, the non-soy, legume, protein material of this disclosure additionally has a Protein Dispersability Index of greater than about 70%. Preferably, the non-soy, legume, protein material comprises at least 20% dry weight pea protein, meets USDA Organic Certification requirements, and is not genetically modified.

### DETAILED DESCRIPTION OF DISCLOSURE

The present disclosure relates to a non-soy, legume, protein material that is at least 50% dry weight non-soy, legume, protein; has a pH of about 4-8; and has a Nitrogen Solubility Index of greater than about 40%. Preferably, the non-soy, legume, protein material of this disclosure additionally has a Protein Dispersibility Index of greater than about 70%. Preferably, the non-soy, legume, protein material of this disclosure comprises at least 20 dry weight pea protein, most preferably at least 80% dry weight pea protein. Preferably, the non-soy, legume, protein material of this disclosure meets USDA Organic Certification requirements and is not genetically modified. Preferably, the non-soy, legume, protein material of this disclosure meets Non-GMO Project Verified requirements. Non-GMO Project Verified is a nonprofit organization offering a third-party Non-GMO verification program as currently disclosed at www.nongmoproject.com.

The present disclosure comprises methods for making the disclosed non-soy, legume, protein material. The method of this disclosure comprises grinding de-hulled, non-soy, legumes; combining the ground matter with water to make an intermediate slurry; removing the insoluble portion (which contains insoluble fiber and starch) of the ground matter in the intermediate slurry; precipitating the protein material from the remaining portion of the intermediate slurry; solubilizing the precipitated protein using acids and/or bases; and treating enzymatically the solubilized protein matter to make the non-soy, legume, protein material of the present disclosure. Optionally, the protein material could be precipitated and then treated with enzymes. Optionally, the protein material of this disclosure is defatted before being ground. The process of this disclosure is not limited by the number of process steps. The resultant non-soy, legume, protein material could be further processed to remove at least a portion of its water content, or further processed so as to be agglomerated with itself and/or with other ingredients. Further processing could comprise solubilization and enzyme hydrolysis.

The non-soy, legume, protein material of this disclosure could then be used to make food products that would have a smooth, creamy mouthfeel with a desired product viscosity. The success of the formulation would be due to the non-soy, legume, protein material of this disclosure, with its sedimentation, dispersibility, solubility, emulsification, stability, and viscosity functions (even at high protein addition levels) desired by product developers. The list of non-soy legume varieties used to make the treated protein material of this disclosure comprise, but are not limited to, peas (e.g., yellow field peas and chickpeas), fava beans, black beans, red beans, lentils, lupin (i.e., lupini, lupin beans) and combinations thereof. The non-soy legume material used to make the non-soy, legume, protein material of the present disclosure may contain no peas. Preferably, the content of the non-soy, legume, protein material of the present disclosure is at least 0, 10, 15, 20, 25, 30, 35, 40, 45, 50, 60, 65, 70, 75, 80, 85, 90, 95, or 99% dry weight non-soy legumes, most preferably at least 0, 10, 15, 20, 25, 30, 35, 40, 45, 50, 60, 65, 70, 75 80, 85, 90, 95 or 99% dry weight protein.

Preferably, the non-soy legume varieties used to produce the non-soy, legume, protein material of this disclosure are not genetically modified, meet Non-GMO Project Verified requirements, are naturally bred, and are not bioengineered. Preferably, the non-soy legume varieties used to produce the non-soy, legume, protein material of this disclosure are Organic Certified according to USDA regulations. Organic Certified means that the source of the ingredients and the finished food product have been produced according to specific requirements wherein the legume plants would only come in contact with program approved herbicides, pesticides, process aids, and cleaning materials.

The non-soy, legume, protein material of this disclosure preferably contains at least 0, 10, 15, 20, 25, 30, 35, 40, 45, 50, 60, 65, 70, 75, 80, 85, 90, 95 or 100% dry weight pea protein material. As used herein, “pea” means the mostly small spherical seed of the pod fruit Pisum sativum. In particular, the pea used in this disclosure is from varieties of the species typically called field peas or yellow peas that are grown to produce dry peas that are shelled from the mature pod. Peas have been harvested as human food as far back as the early third century BC. Peas are traditional foods in the diets of people living on every continent, most particularly in Europe, Asia, North Africa, and North America. Though traditionally a cool-season crop, new varieties have been bred that can be grown in hotter climates and in dryer climates. Peas also have been bred to contain higher and higher protein content. These breeding practices, as well as the cultural eating histories of so many people, make peas an excellent source for protein for many consumers worldwide.

All percentages are in dry weight, unless specified otherwise as total weight. High water content foods are edible products (i.e. human or animal food) containing greater than 20% total weight water. High protein content foods contain greater than 4% dry weight protein. As a comparison, cow's milk contains 3-4% total weight protein.

The non-soy, legume, protein material of this disclosure comprises at least 50% dry weight protein, preferably at least 80% dry weight protein. Non-soy legumes (as traditionally harvested and dried), have a hull portion (about 6-10% dry weight of whole non-soy legume) and a seed portion (about 90-94% dry weight of whole non-soy legume). For example, when the non-soy legume is peas, the hull portion is about 6-10% dry weight of whole peas and the seed is about 90-94% dry weight of whole peas. When the pea hull is removed, the pea seed portion has a content of up to about 12-15% total weight moisture, about 50-60% total weight starch, about 2-4% total weight fat, and about 10-30% total weight protein. The product of this disclosure is not limited by the specific protein content of the peas or by the specific protein content of any other non-soy legume used in the production of the non-soy, legume, protein material of this disclosure. The product of this disclosure is not limited by the specific fiber, starch, or oil content of the non-soy legume variety used in the production of the non-soy, legume, protein material of this disclosure.

Creamy mouthfeel means that a protein example in water or in a food product would have a smooth and non-gritty (no noticeable particles present) feel in the mouth, while also having some thickness that coats the tongue and mouth surfaces. Gritty (also called grainy) mouthfeel means that the tongue and/or mouth surfaces can feel tiny particles. Creamy appearance means that the sample or product appears smooth and homogeneous. Gritty (or mealy) appearance means that the product appears rough and/or heterogeneous. Sedimentation and separation appearance means that the sample or product appears to be in layers, usually one layer darker or more opaque than another layer. Thickness refers to how a sample or product moves when force is applied to it. More movement means less thick. The term thicker means more viscous. Pourable means that when a container of product is tilted to the side, the product in the container moves. Cuttable and spoonable mean that a utensil can create a clean break in a contained mass of product when the utensil is used to cut a piece off of the product mass, or when the utensil is used to scoop out a portion of the product mass.

Most non-soy, legume, proteins have some functionality (e.g., bulking, thickening, emulsification, foam stabilizing) when in contact with water. At least in part these functions are based on the interaction of the protein with water, that is, an interaction caused by the protein having both charged and uncharged, or polar and nonpolar, or hydrophobic and hydrophilic areas in its amino acid molecular structure (that is, its strand or molecular chain of amino acids). These areas of the protein interact with water, which also has both polar and non-polar areas in its structure. Water also interacts with many materials, causing those materials to change into charged solute forms when they are in a water solution. Being charged, those solutes can also interact with proteins. Changing the physical structure (such as unraveling the folded and twisted structure of protein strands) or the physical composition (such as by breaking off amino acids or by chemical reactions with protein's amino acids) of non-soy, legume, protein materials can alter the functionality of the non-soy, legume protein materials. Alterations can be in both in type and amount of functionality.

The non-soy, legume, protein material of the current disclosure has increased functionality over other non-soy, legume, protein materials due to the process treatment used to make the non-soy, legume, protein material of this disclosure. The process (including the enzymatic treatment) at least partially unravels the protein structure, exposing charged and uncharged amino acids that were previously tied-up and/or hidden in the interior of the protein strand structure. The heat, acid, alkali, and enzyme usage in the protein separation process of the present disclosure is not such that it would have created a significant amount of peptide bond breakage that would have led to the release of free amino acids and/or small protein strands. This is different from the acid, alkali, and enzymatic process treatments often used to make the non-soy, legume, protein materials currently available to product developers. An example of an available enzyme treated protein material is example 870H (from Puris, Minneapolis, Minn.). Example 870H is produced with a protease enzyme hydrolysis so as to give it more solubility than Example 870 (from Puris, Minneapolis, Minn.), which has had no enzyme treatment.

The enzyme treatment used in the process of the present disclosure is a protein-glutaminase enzyme treatment. Protein-glutaminase deamidates turning glutamine it into glutamic acid, and in doing such, under the other conditions of the process of the present disclosure, the protein strand at least partially unravels. Such physical change occurs without breaking the amino acid bonds of the non-soy, legume protein backbone that would cause release amino acids from the protein strand, and without altering the size of the non-soy, legume, protein strand.

A continuing challenge in the plant protein material market is the control of the protein material's solubility properties and the mouthfeel of the protein material while it is in solution and in food products, especially at high protein content levels. Control of solubility means control of several protein functions, comprising, but not limited to, sedimentation, dispersibility, emulsification, and foam stability. Currently, many marketed plant protein materials have limited solubility, and as such, those protein materials could fall out of solution and precipitate at high content levels. Alternatively, many currently marketed plant protein materials absorb so much water while in solution and in food products that the solution or food products are too viscous for processing or for consumption. For example, with RTD beverages, with the currently marketed proteins, at a high protein content level (e.g., 20% protein) a finished RTD product could be too thick to process or to consume because it would be pudding-like in texture and as such too thick to pump or to pour from a container.

The gritty texture of some plant protein materials when in water can be from several causes. Currently, many marketed plant protein materials coagulate and/or precipitate when heated during the processing of the protein material and/or when heated in the production of a finished food product. Acid and alkali treatment during protein material processing can also cause those proteins to precipitate or coagulate, which could also create undesirable gritty mouthfeel.

The gritty texture of some plant protein materials in water could be from finished protein material product particle size. As already discussed, particle geometry can influence how the tongue perceives product particles. Currently many marketed plant protein materials have particle size distributions that contain large enough particles present that a consumer's tongue can perceive them as grit. If the particles are three-dimensional (i.e., semi-spherical), then by theory, those larger protein material particles could be perceived as grit. If the overall texture of the protein solution (or high water content food product) is less viscous (i.e., thin) then the tongue would be able to feel the grit more easily than if the protein solution were more viscous. Example 870H (PURIS, Minneapolis, Minn.) was made using protease enzyme treatment on pea protein matter. 870H has a lower viscosity than the pea based non-soy, legume, protein material of the current disclosure (Example Protein 2.0), and the protein particles are more noticeable in 870H than in Protein 2.0 (See Table 2).

There is a need for a non-soy, legume, protein material with modified physical characteristics that would allow the modified non-soy, legume, protein material to have the water solubility properties and the creamy mouthfeel necessary to allow product formulators to create acceptable food products in a wide range of protein content levels. The non-soy, legume, protein material of the current disclosure has the physical characteristics that allow high contents of protein without the resulting viscosity becoming too thick for processing or becoming too thick for consumption, such as with a beverage food product.

The creators of the non-soy, legume, protein material of the current disclosure found a process for creating an improved non-soy, legume, protein material, wherein the improved protein material of this disclosure has solubility as shown by physical testing (Centrifuge Sedimentation Test [Test A], NSI [Test B], and PDI [Test C]) and organoleptical properties as shown by sensory testing (Sensory Testing [Test D]), such that high non-soy, legume, protein material content levels in food products can be achieved with resulting acceptable physical and organoleptical characteristics. Examples Pea Milk, Ready-To-Drink (RTD) Beverages, Dry Beverage Blends (DBB), Cream Cheese, and Yogurt are provided as Examples of product formulas that can use the non-soy, legume, protein material of the present disclosure to boost finished product protein material content while creating food products with consumer desired texture, flavor, and color. The present disclosure is not limited by the specific formulas written in the tables of this disclosure document. This disclosure has within its scope any formula for food products such as, but not limited to, beverages, sauces, cheese analogs, meat analogs, egg analogs, extruded products, and other protein containing food products that could use the non-soy, legume, protein material of the current disclosure as at least part of the source of protein in those food products. This disclosure also has within its scope any formula for supplements, pharmaceuticals and industrial products that could use the non-soy, legume, protein material of this disclosure as at least part of the source of protein in those products.

Native pea proteins (that is, as traditionally grown, harvested, and ground), and other non-soy legumes, have an isoelectric point of about pH 4.5. The isoelectric point is the pH at which a particular molecule carries no net electrical charge in the statistical mean. This means that pea proteins (which are predominantly made up of globulin proteins) have a minimum solubility near the isoelectric point of pH 4.5 and a high solubility above and a moderate solubility below pH 4.5. Changes in the availability of protein's amino acids to interaction with water (e.g., due to acid, alkali, and/or enzyme treatment) can change the isoelectric point of a non-soy, legume, protein material.

Native pea proteins contain another group of proteins, here called albumins or whey proteins. These albumin proteins are more water soluble than the globular proteins. Most commercially available non-soy, legume, protein materials are composed of the globular form of protein, whether the proteins were separated from starch and fiber legume seed portions via acid or alkali processing. After the globular proteins are coagulated and precipitated (through acid and/or alkali addition), the globular proteins are physically separated from the albumin proteins and other soluble materials (e.g., small chain carbohydrates) through filtration and/or centrifugation. In an embodiment of this disclosure, the albumin non-soy, legume, proteins are combined with the globular non-soy, legume, proteins either before or after enzyme treatment of the globular non-soy, legume, proteins material in order to make a finished non-soy, legume, protein material of the present disclosure.

Proteins (globular form) are made up of a bundle of molecules of different lengths, each molecule (i.e., strand) having amino acids with neutral and charged reactive points along their lengths. Native (globular form) proteins have a non-linear, folded or twisted structure wherein sections of protein strands fold back along themselves. This folding back causes some charged amino acids to be buried within the protein mass structure. Sometimes amino acids along the protein strands react with each other where the strands fold back along themselves. The protein neutral and charged reactive points allow proteins to react with water, chemicals in the water, solutes in the water, enzymes in the water, and other proteins in the water. If a protein is not charged at its isoelectric point of pH 4.5, then that protein is at its least interactivity with water at that pH of 4.5.

The creators of the current disclosure discovered a process that allows them to alter the structure of non-soy, legume, proteins such that the non-soy, legume, protein in the disclosed non-soy, legume, protein material has an increased water solubility, improved flavor, aroma, and color, and improved mouthfeel in water (e.g., non-gritty, creamy). This improved functionality leads to positive protein material characteristics comprising, but not limited to, reduced sedimentation, increased NSI (Nitrogen Solubility Index), increased PDI (Protein Dispersibility Index), decreased gritty mouthfeel, increased creamy mouthfeel, decreased perceived saltiness, decreased perceived bitterness, and decreased perceived cooked pea flavor.

The non-soy, legume, protein material of this disclosure is produced under processing conditions that give the non-soy, legume, protein material a pH range of about 4-8. The processing conditions used to adjust the pH of the non-soy, legume, protein material can be done by various methods known in the art, e.g., the addition of acid and/or base during separating of the protein from the fiber and starch portions of the native legume, or the addition of acid and/or base after the separation of the protein from the fiber and starch portions of the native legume, or the addition of acid and/or base after reduction of water from the protein portion of the starting ground non-soy legume material. The key is a resulting pH in the range of about 4-8, preferably in the range of about 6-8. The protein in non-soy legumes comprises many individual proteins of various molecular weights. To make non-soy, legume protein more soluble, it can be treated in such a way as to break some of those protein molecules into smaller molecules exposing more charged and reactive amino acid sites for greater interaction with water molecules. Some amino acids could be completely cleaved from the protein strand. This is commonly called hydrolyzing the protein. The resulting hydrolyzed proteins are commonly called protein hydrolysates. The hydrolyzation can be done by alkali and/or acid and/or enzyme addition during the processing of the protein matter into protein material. Alkali and acid addition can break protein strands into smaller units by attacking amino acid to amino acid bonds along the protein strand. Enzymes, such as proteases, can also cleave amino acid to amino acid bonds along the protein strand. Cleaving a protein strand along its length creates more end of strand amino acids, hence increasing the total protein mass's interaction with water. Too much protein reaction with alkali, acid, and/or certain enzymes (such as proteases) could go too far, break too many amino acid-amino acid bonds, and actually reduce the protein mass's interactivity with water. That would decrease the overall functionality of the protein mass.

Another challenge of breaking the non-soy, legume, protein strand into smaller molecular weight pieces could be the creation of bitter flavor notes and gritty mouthfeel. When breaking a legume protein into smaller molecular weight strands, additional amino acids could become exposed to interaction with taste buds. Also, enzyme (e.g., proteases) reactivity with legume proteins could also create free amino acids that could have been cleaved from the legume protein strand. Though these protein strand terminal amino acids will increase the reactivity of the protein with water molecules, and thus increase protein solubility, that increased solubility will be at the expense of additional metallic or bitter flavors. The amino acids (free amino acids and terminal amino acids) can be now available to interact with sensory sites on the tongue and mouth. They can create perceived metallic and/or bitter flavors. This is a difficult trade-off for product developers choosing proteins for their food product formulations. The parties of this disclosure have found a better way to create more functionality in non-soy, legume, protein materials without trading the increased solubility for poorer flavor or texture.

The process for producing the non-soy, legume, protein material of this disclosure contains two broad process steps: 1) creating a non-soy, legume, protein material intermediate slurry containing at least 50% dry weight protein; and 2) treating the non-soy, legume, protein material intermediate slurry so as to create a unique enzyme treated non-soy, legume, protein material with improved solubility, flavor, aroma, color, and mouthfeel (e.g., non-gritty and creamy). As already discussed, the improved solubility of the non-soy, legume, protein of this disclosure means increased solubility, which in turn leads to increase functionality in the form of, but not limited to, greater ability to disperse solids, to suspend solids, to create emulsions, to stabilize foams, and to create greater viscosity. This last functionality is of particular use in high water content products such as soups, sauces, milks, and beverages where thickness is wanted, but not such thickness at high protein content levels that a food product is too thick to flow in pipes and pumps, and not too thick to pour and/or drink. Also, as already discussed, at high levels of protein content, if all of the protein in a food product is not dissolved, the non-dissolved protein could be perceived as grit. Also, as already discussed, if the non-soy, legume, protein material is in non-dissolved particles, those particles that are not maintained in a colloidal suspension could be perceived visually as gritty or mealy and perceived by the tongue as grit. If the particles are large enough to be seen, then the tongue could perceive the particles as grit.

Producing an at least 50% dry weight protein non-soy, legume, protein intermediate slurry from non-soy legumes (e.g., peas) could be done by several different processes known by those who practice in this art. The specific method chosen does not limit the scope of this disclosure. In general, the process comprises reducing the non-soy legume into particles that could then be separated into fiber, starch, and protein portions.

In one embodiment of the present disclosure, one method of such separation can be to grind the dry non-soy legumes and then use a series of air classification steps to remove the lighter weight fiber and starch particles, leaving behind an intermediate non-soy, legume, protein matter that has at least 50% dry weight protein content.

In another embodiment of the present disclosure, a second method of separation can be to grind the non-soy legumes so as to only remove the hull; then grind the remaining non-soy legume matter with enough water to create an intermediate stage slurry; and then separate out the insoluble fiber and starch portions from the intermediate stage slurry so as to create a non-soy, legume, protein intermediate slurry containing the soluble protein portion. At this point the protein portion contains both globular and albumin protein forms. Separation of non-soy, legume, protein portion from the intermediate stage slurry in this second method could be done using various separation techniques. These techniques comprise causing the globular protein form to coagulate and precipitate out of the intermediate stage slurry protein portion, which would allow the separation of the protein precipitate from the soluble portion (e.g., albumin proteins, ash, and small carbohydrates) by, but not limited to, use of decanters, centrifuges, clarifiers, hydro cyclones, and combinations of such. The finished non-soy, legume, protein material could be created by removing at least a portion of the water content through various separation techniques comprising, but not limited to, use of decanters, centrifuges, clarifiers, ovens, spray dryers, fluid bed dryers, drum dryers, and combinations of such.

During the separation of protein from the non-soy legume intermediate stage slurry, some of the protein would precipitate out of the intermediate stage slurry due to changes in pH of the slurry. Some of the protein in the starting legume matter could remain soluble even at that pH. As already discussed, this soluble protein is often called albumin (or whey) and it has a composition and physical properties different from that of the precipitated protein (globular protein). Using peas as a non-soy legume example, one difference between the two legume (e.g., peas) protein portions is their amino acid profiles, which differ in sulfur containing amino acid content. When combined in appropriate portions, the resulting combined globular and albumin pea protein material could have the amino acid content and protein digestibility necessary to have a calculated PDCAAS of 0.75-1.0. This is the PDCAAS of milk proteins, which are considered in the market to be “complete proteins”. The means of calculating the PDCAAS of a protein is explained on the FDA.gov website. One embodiment of the present disclosure is a non-soy, legume, protein material, wherein the protein material contains globular and albumin proteins and has a PDCAAS of 0.75-1.0. Another embodiment of the present disclosure is a pea protein material, wherein the pea protein material contains globular and albumin proteins and has a PDCAAS of 0.75-1.0.

In an embodiment of the current disclosure, the non-soy, legume, protein material contains more than one form of protein, more than one source of protein, and combinations thereof. In an embodiment of the current disclosure, a non-soy, legume, protein material has at least 70% of its protein in globular form and at least 5% of its protein in albumin form. Preferably the non-soy, legume protein material has a PDCAAS of 0.75-1.00.

In an embodiment of the current disclosure, the non-soy, legume, protein material has at least 65% of its protein from non-soy legumes, and at least 5% of its protein from nuts (e.g., almonds), grains (e.g., rice), vegetables (e.g. broccoli), fruits (e.g., avocados), or combinations of such. Preferably the non-soy, legume protein material has a PDCAAS of 0.75-1.00.

In one embodiment of the present disclosure, the non-soy, legume, protein material of the present disclosure comprises a combination of globular non-soy, legume, protein and albumin non-soy, legume, protein in such portions as to create a non-soy, legume, protein material with a PDCAAS of about 0.75-1.00.

In an embodiment of this disclosure, a non-soy, legume, protein material containing at least 50% dry weight protein is made by the second method already described. The non-soy, legume, protein is separated from the intermediate slurry (made by the second method) by adjusting the slurry to the non-soy, legume, protein's isoelectric point causing the protein to coagulate. The coagulated protein is then removed from the bulk of the intermediate slurry and the pH of the coagulated protein is adjusted to about pH 4-8 using a food grade buffer comprising, but not limited to, calcium hydroxide, potassium hydroxide, sodium hydroxide, and combinations thereof. Enzymes could be added to the neutralized non-soy, legume, protein material at this point in the process.

In one embodiment for a process of this disclosure, the albumin protein portions can be combined with the non-neutralized globular protein before or after further process treatments, such as enzyme treatment. In an embodiment of this disclosure, the precipitated non-soy, legume, protein material, after separation from insoluble fiber and starch legume matter portions, is further treated with enzymes to make the non-soy, legume, protein more soluble, and being such, more functional in terms of, but not limited to, dispersability, emulsifying, and viscosity building. In an embodiment of this disclosure, the enzymes used to treat the precipitated non-soy, legume, protein material are at least in part protein-glutaminase.

Protease enzymes have been used to cleave non-soy, legume, protein peptide bonds to reduce protein strand size. This decreased protein strand size, along with the resulting increased number of charged end amino acids, could make the enzyme treated protein more reactive with water, thus more soluble. But, as already discussed, the enzyme treated non-soy, legume, protein material could have a bitter flavor and, often, a gritty texture. The enzymes used to reduce protein strand size would be endo-protease, exo-protease, or combinations. Enzymes used could comprise, but not be limited to, Chymotrypsin, Trans gluaminase, and Peptidoglutaminas from Bacillus circulans.

In an embodiment of this disclosure the non-soy, legume, protein material of this disclosure is made using a protein-glutaminase enzyme to at least partially hydrolyze non-soy, legume, protein in the non-soy, legume, protein material.

Protein-glutaminase is a bacterial strain of Chryseobacterium proteolyticum. Not to be limited by theory, the protein-glutaminase enzyme hydrolyzes the amino group of glutamine residues in non-soy, legume proteins that are in the non-soy, legume, protein material. In this process of hydrolysis, glutamine is converted to glutamic acid. Furthermore, not to be limited by theory, deamination of glutamate by the protein-glutaminase enzyme could significantly change the tertiary structure of the non-soy, legume protein in the non-soy, legume, protein material exposing more amino acids to interaction with water, thus allowing greater interaction with water, and so greater protein solubility. The protein-glutaminase enzyme would not cleave the protein creating smaller protein strands, but it would react with glutamine residues and open up the protein structure to expose the hydrophobic folding. In general, when protein-glutaminase is converting the glutamine residues to glutamic acid, the negative charge on the protein mass increases as the negatively charged carboxyl groups are increased. The increase in negative charges on the protein strand causes depression in the isoelectric point and increases the non-soy, legume, protein's hydration ability. The hydrolysis also increases the repulsion between non-soy, legume, protein molecules causing improvement (i.e., increase) in non-soy, legume, protein material solubility. The hydrolysis exposes the protein's hydrophobic structure that was concealed in the interior of the protein, and improves the amphiphilic nature of the protein by change in the higher order structure that could improve the non-soy, legume, protein's emulsification ability, suspension stability, and foamability.

In an embodiment of the current disclosure, a non-soy, legume, protein material could be produced by treating non-soy, legume, proteins with protease enzymes and with protein-glutaminase enzymes, simultaneously or sequentially. This double enzymatic action could cause increased non-soy, legume, protein material solubility through reduction in protein strand size, creation of end amino acids, and creation of more open protein strand structure. The protease enzymes, though, could reduce the activity of the protein-glutaminase enzymes because the protease could attack the protein-glutaminase itself.

In an embodiment of the current disclosure, a non-soy, legume, protein material could be produced by treating whole or ground non-soy legumes, fully or partially hydrated, with enzymes before, during, or after pH adjustments. Such enzymes could comprise proteases and/or protein-glutaminase.

In an embodiment of the current disclosure, the process of making a non-soy, legume, protein material comprises the steps of: a) grinding de-hulled non-soy legumes to make a ground non-soy legume matter; b) mixing the ground non-soy legume matter with water to make an intermediate slurry; c) separating the insoluble fiber and starch portions from the soluble protein portion of the intermediate slurry to make a intermediate protein portion slurry; d) coagulating protein in the intermediate protein portion slurry; e) removing the coagulated protein from the intermediate protein portion slurry and solubilizing the protein in water; f) neutralizing the coagulated protein solubilized in water to make a neutralized protein slurry; g) intermixing the neutralized protein slurry with enzyme material; h) heating the neutralized protein slurry containing enzyme to about 32 C-121 C for 5 minutes-6 hours; and i) removing water from the heated neutralized protein slurry to make a non-soy, legume, protein material that in solution with water and in food products creates a smooth, creamy, non-gritty texture without cooked vegetable, bitter, and/or metallic flavors. The process comprises the use of a deaminating agent, such as an enzyme, wherein the enzyme used is a bacterial strain of Chryseobacterium proteolyticum, including, but not limited to, protein-glutaminase.

In an embodiment of the current disclosure, the process comprises a heating of the neutralized protein slurry containing enzyme from 32 C-65 C. In an embodiment of the current disclosure, the heating of the neutralized protein slurry containing enzyme is for 5 minutes-130 minutes. In an embodiment of the current disclosure, the heating of the neutralized protein slurry containing enzyme is done in at least two heating processes, of which one is at least at 93 C.

In an embodiment of the current disclosure, a non-soy, legume protein material could be produced with a process that comprises at least two process steps that heat the non-soy, legume protein matter to over about 93 C before protein-glutaminase enzyme addition to the protein matter and then an additional heating step wherein the protein matter with protein-glutaminase enzyme is heated to over about 93 C. Preferably the heating steps are completed utilizing steam direct or indirect cooking, drum drying, spray drying, convection heating, kettle cooking, microwave heating, or combination thereof.

Protein-glutaminase enzymes were explored by the parties of the present disclosure as a means of increasing the functionality of non-soy, legume, proteins without the creation of unwanted flavors, colors, and textures. The functionalities wanted comprised the ability of the resulting protein to create product viscosity, but in moderation, so as to allow for high protein usage levels in food products such as (but not limited to) beverages—without grittiness. Creamy texture was desired. The protein-glutaminase enzyme was sourced from Amano Enzyme. The protein-glutaminase enzyme is disclosed and discussed in U.S. Pat. Nos. 7,279,298 and 7,569,378 (Amano Enzyme). Though these two patents describe the creation and general use of protein-glutaminase enzyme, these patents do not disclose the full process conditions to create the desired final non-soy, legume, protein material composition of the present disclosure.

Both time and temperature conditions during protein-glutaminase enzyme hydrolysis of non-soy, legume, proteins are important towards making the non-soy, legume, protein material of the present disclosure. Of course, the process conditions that created the coagulated and precipitated protein (with or without albumin protein) to which the enzyme is applied is also important towards the making of the highly functional non-soy, legume, protein material of the present disclosure.

TABLE 1
Bench Trials: Protein-Glutaminase
Addition Levels and Reaction Times

| Enzyme Usage | Time | Observations |
|---|---|---|
| 0.05-4%. | 10 min.-6 hr. | Significant reduction in pea/cooked |
| | | flavor & creamy texture |
| 0.01-1% | 10 min-6 hr. | Slight reduction in pea/cooked |
| | | flavor & creamy texture |

Table (1) illustrates tasting evaluation of non-soy, legume, protein material made using protein-glutaminase and pea protein using different levels of protein-glutaminase held at different reaction times at about 32 C-65 C. This bench work was used towards making the decisions on the range of enzyme usage and the enzyme treatment process time in the process of the current disclosure, taking into account other process elements also (e.g., heat, pH).

In an embodiment of this disclosure of the process to make the disclosed non-soy, legume, protein material, the temperatures and times used during the treatment of the protein with enzyme is about 32 C-65 C, preferably about 46 C-60 C, for about 5 minutes-6 hours, preferably 5-130 minutes. The inventors found that conditions outside these ranges could create too little or too much hydrolysis of the amino group of glutamic residues in the non-soy, legume, protein that would affect the functional characteristics of the resulting non-soy, legume, protein material. To end the enzymatic hydrolysis activity, the non-soy, legume, protein material was heated to over 93 C. The pea protein material could then be left liquid or reduced to less than about 25% water content. The coagulated, precipitated protein used for reaction with protein-glutaminase could be pasteurized or not pasteurized; could be homogenized or not homogenized; could be dried and then solubilized or not dried and solubilized; could contain globular and albumin proteins or contain only globular proteins or contain only albumin proteins; could be at least partially below its isoelectric point, at its isoelectric point or above its isoelectric point; or combinations of such at the time the protein-glutaminase is combined with the protein for at least some hydrolysis of the protein at a temperature of about 32 C-121 C.

The water reduction method used in the present disclosure is not limited in the production method of the highly functional non-soy, legume, protein material of this disclosure. Such water reduction process could comprise, but would not be limited to, spray drying, fluid bed drying, oven drying, drum drying, convection drying, vacuum drying and freeze drying. The non-soy, legume, protein material of the present disclosure can be dried by spray drying using an inlet slurry temperature of about 32 C-121 C to dry the non-soy, legume, protein material at about 93 C-315 C.

Spray drying conditions, such as nozzle configuration and solids content of the non-soy, legume, protein material going to the spray dryer, could have an effect on the particle size of the finished dried protein material. Lower solids content of the protein material going to the spray drier could produce a dried protein material with a smaller particle size versus protein material spray dried using a higher solids material. Dried protein material that has particles of smaller particle size could be perceived to have a smoother mouthfeel then dried protein material with larger particle size. A finer mist created by smaller nozzle configuration could assist in creating finer spray droplet size, which would lead to dried material particles of smaller size.

Protein structure could affect the geometry of the resulting dried non-soy, legume, protein particles. The spray dried particles of the protein-glutaminase hydrolyzed pea protein material of the current disclosure created a creamy, non-gritty texture. Whereas, the spray dried particles of the protease hydrolyzed pea protein material could create a gritty, less creamy texture, such as with example P870H (PURIS). The experimental example Protein 2.0 (P 2.0) of the current discloser had a smooth, creamy texture without metallic or bitter flavor. The example was hydrolyzed with protein-glutaminase. Not to be limited by theory, the protein-glutaminase unfolded at least some of the pea protein strands through the enzyme's conversion of glutamine to glutamic acid. Protein-glutaminase did not shorten the protein strand length or reduce the protein strand molecular weight because protein-glutaminase deamidates the protein without reducing the protein chain length by cutting the peptide bonds. Theoretically, this conversion of glutamine to glutamic acid would cause the protein strands to unravel and straighten out, which could cause flatter, more platelet shaped particles upon drying. Hydrolysis of protein using protease enzymes could cause more granular, less soluble spray dried protein structure due, theoretically, to interaction between protein strands and/or between portions of the protein strand.

In an embodiment of the present disclosure, the non-soy, legume, protein material of this disclosure may be used in any food product, e.g., but not limited to beverages, extruded snacks, bakery products, confectionery products, meat or meat-analog products, dairy or dairy-alternatives, cheese or cheese-alternative products, beverages, and sauces.

In an embodiment of this disclosure, the non-soy, legume, protein material is in a food product, wherein the non-soy, legume, protein material is at least 1, 5, 10, 15, 20, 25, 30, 35, 40, 45, 50, 55, 60, 65, 70, 75, 80, 85, 90, 95, or 99% dry weight of the food product.

In an embodiment of this disclosure the non-soy, legume, protein material is used in making a high moisture food product, wherein the high moisture food product is a beverage or sauce selected from the group comprising milks, sports drinks, nutritional beverages, fruit based beverages, carbonated beverages, non-carbonated beverages, non-dairy beverages, acidified hot-fill beverages, Ready-To-Drink beverages, retorted beverages, aseptic packed beverages, sauces, gravies, sweet and sour sauces, fermented base sauces (e.g., oyster sauce, soy sauce, teriyaki sauces), broths, tomato based sauces, soups, white sauces, and combinations thereof.

In an embodiment of this disclosure the non-soy, legume, protein material of this disclosure is used in a beverage, preferably at greater than about 1, 5, 10, 12, 15, 20, 25, 30, 40, 50, 60, 70, 80, 90, 95, or 99% total weight of the beverage, most preferably at 1, 5, 10, 12, 15, 20, 25, 30, 40, 50, 60, 70, 80, 95, or 99% dry weight of the beverage.

In an embodiment of this disclosure the non-soy, legume, protein material of the current disclosure is used in beverage food products with additional ingredients comprising, but not limited to hydrating, fluidizing, texturizing, bulking, flavoring, emulsifying, sweetening, and stabilizing ingredients and combinations thereof. These additional ingredients comprise, but are not limited to fats, oils, glycerin, polyols, sugars, syrups, spices, salts, acids, alkalis, starches, fibers, other proteins (e.g., albumin, globulins), hydrocolloids, methylcellulose, carbohydrates, and celluloses.

In an embodiment of this disclosure the non-soy, legume, protein material of this disclosure is used in a sauce, preferably at greater than about 1, 5, 10, 12, 15, 20, 25, 30, 40, 50, 60, 70, 80, 90, 95, or 99% total weight of the sauce, most preferably at 1, 5, 10, 12, 15, 20, 25, 30, 40, 50, 60, 70, 80, 90, 95, or 99% dry weight of the sauce.

In an embodiment of this disclosure non-soy, legume, protein material is used in sauce food products with additional ingredients comprising, but not limited to hydrating, fluidizing, texturizing, bulking, flavoring, emulsifying, sweetening, and stabilizing ingredients and combinations thereof. These additional ingredients comprise, but are not limited to fats, oils, glycerin, polyols, sugars, syrups, spices, salts, acids, alkalis, starches, fibers, other proteins (e.g., albumin, globulins), hydrocolloids, methylcellulose, celluloses, carbohydrates, and combinations thereof.

In an embodiment of this disclosure the non-soy, legume, protein material is used in dairy and non-dairy (i.e., dairy analogs, dairy alternatives) food products, preferably at greater than about 1, 5, 10, 12, 15, 20, 25, 30, 40, 50, 60, 70, 80, 90, 95, or 99% total weight of the food product, most preferably at 1, 5, 10, 12, 15, 20, 25, 30, 40, 50, 60, 70, 80, 90, 95, or 99% dry weight of the food product.

In an embodiment of this disclosure the non-soy, legume, protein material of the current disclosure is used in dairy and non-dairy (i.e., dairy analogs, dairy alternatives) food products with additional ingredients comprising, but not limited to hydrating, fluidizing, texturizing, bulking, flavoring, emulsifying, sweetening, and stabilizing ingredients and combinations thereof. These additional ingredients can comprise, but are not limited to fats, oils, glycerin, polyols, sugars, syrups, spices, salts, acids, alkalis, starches, fibers, other proteins (e.g., albumin, globulins), hydrocolloids, methylcellulose, celluloses, carbohydrates, and combinations thereof.

In an embodiment of this disclosure the non-soy, legume, protein material of this disclosure is used in extruded or textured protein food products, preferably at greater than about 1, 5, 10, 12, 15, 20, 25, 30, 40, 50, 60, 70, 80, 90, 95, or 99% total weight of the food product, most preferably at 1, 5, 10, 12, 15, 20, 25, 30, 40, 50, 60, 70, 80, 90, 95, or 99% dry weight of the food product.

In an embodiment of this disclosure the non-soy, legume, protein material of the current disclosure is used in cheese and non-cheese (i.e., cheese analogs, cheese alternatives) food products with additional ingredients comprising, but not limited to hydrating, fluidizing, texturizing, bulking, flavoring, emulsifying, sweetening, and stabilizing ingredients and combinations thereof. These additional ingredients can comprise, but are not limited to phosphates, citrates, silicates, fats, oils, glycerin, polyols, sugars, syrups, spices, salts, acids, alkalis, starches, fibers, other proteins (e.g., albumin, globulins), hydrocolloids, methylcellulose, celluloses, carbohydrates, and combination thereof.

In an embodiment of this disclosure the non-soy, legume, protein material of this disclosure is used in cheese and non-cheese (i.e., cheese analogs, cheese alternatives) food products, preferably at greater than about 1, 5, 10, 12, 15, 20, 25, 30, 40, 50, 60, 70, 80, 90, 95, or 99% total weight of the food product, most preferably at 1, 5, 10, 12, 15, 20, 25, 30, 40, 50, 60, 70, 80, 90, 95, or 99% dry weight of the food product.

In an embodiment of this disclosure the non-soy, legume, protein material of this disclosure is used in meat and non-meat (i.e., meat analogs, meat alternatives) food products, preferably at greater than about 1, 5, 10, 12, 15, 20, 25, 30, 40, 50, 60, 70, 80, 90, 95 or 99% total weight of the food product, most preferably at 1, 5, 10, 12, 15, 20, 25, 30, 40, 50, 60, 70, 80, 90, 95, or 99% dry weight of the food product.

In an embodiment of this disclosure the non-soy, legume, protein material of the current disclosure is used in meat or non-meat (i.e., meat analogs, meat alternatives) food products with additional ingredients comprising, but not limited to hydrating, fluidizing, texturizing, bulking, flavoring, emulsifying, sweetening, and stabilizing ingredients and combinations thereof. These additional ingredients can comprise, but are not limited to fats, oils, glycerin, polyols, sugars, syrups, spices, salts, acids, alkalis, starches, fibers, other proteins (e.g., albumin, globulins), hydrocolloids, methylcellulose, celluloses, carbohydrates, and combinations thereof.

In an embodiment of this disclosure the non-soy, legume, protein material of this disclosure is used in egg and non-egg (i.e., egg analogs, egg alternatives) food products, preferably at greater than about 1, 5, 10, 12, 15, 20, 25, 30, 40, 50, 60, 70, 80, 90, 95, or 99% total weight of the food product, most preferably at 1, 5, 10, 12, 15, 20, 25, 30, 40, 50, 60, 70, 80, 90, 95, or 99% dry weight of the food product.

In an embodiment of this disclosure the non-soy, legume, protein material of the current disclosure is used in egg or non-egg (i.e., egg analogs, egg alternatives) food products with additional ingredients comprising, but not limited to hydrating, fluidizing, texturizing, bulking, flavoring, emulsifying, sweetening, and stabilizing ingredients and combinations thereof. These additional ingredients comprise, but are not limited to fats, oils, glycerin, polyols, sugars, syrups, spices, salts, acids, alkalis, starches, fibers, other proteins (e.g., albumin, globulins), hydrocolloids, methylcellulose, celluloses, carbohydrates, and combination thereof.

In an embodiment of this disclosure the non-soy, legume, protein material of this disclosure is used in extruded or textured protein food products, preferably at greater than about 1, 5, 10, 12, 15, 20, 25, 30, 40, 50, 60, 70, 80, 90, 95, or 99% total weight of the food product, most preferably at 1, 5, 10, 12, 15, 20, 25, 30, 40, 50, 60, 70, 80, 90, 95, or 99% dry weight of the food product.

In an embodiment of this disclosure the non-soy, legume, protein material of the current disclosure is used in extruded or textured food products with additional ingredients comprising, but not limited to hydrating, fluidizing, texturizing, bulking, flavoring, emulsifying, sweetening, and stabilizing ingredients and combinations thereof. These additional ingredients comprise, but are not limited to fats, oils, glycerin, polyols, sugars, syrups, spices, salts, acids, alkalis, starches, fibers, other proteins (e.g., albumin, globulins), hydrocolloids, methylcellulose, celluloses, carbohydrates, and combination thereof.

In an embodiment of this disclosure the non-soy, legume, protein material of the current disclosure is used in extruded of textured food products such as, but not limited to, textured pea protein, extruded snacks or cereal, expanded snacks or cereal, puffed products, extruded meat analogs or alternatives, pasta, noodles, macaroni, and combinations thereof.

In an embodiment of this disclosure the non-soy, legume, protein material is used in making food products wherein some part of the food product production process comprises the making of a high water content intermediate product.

### Examples: Non-Soy, Legume, Protein Material

A non-soy, legume, protein material example, in accordance with the present disclosure was produced using peas that had about 70-90% dry weight pea protein, of which 10-35% dry weight protein was soluble in water at ambient temperature and had a pH of about 4-8. The pea protein material was non-GMO (that is, a non-genetically modified organism). The pea protein material was produced by grinding de-hulled peas with water; creating an intermediate stage slurry of ground pea matter with water; separating insoluble fiber and starch from soluble protein portion in the intermediate stage slurry using centrifugation; coagulating protein in the protein portion; separating and solubilizing the coagulated protein in water; neutralizing the solubilized protein and water pH to about pH 5-8 by adding a food grade buffer; treating the neutralized protein with enzymes; and then heating and drying the resulting pea protein material to about 10-25% water content. The enzyme used for Example #3 was a protein-glutaminase. The enzyme used for Example #2 was a protease. Example #1 had no enzyme treatment. Enzyme treatment comprised a hold time at a specific temperature after enzyme is mixed in with the neutralized protein and water mixture.

Table 2 illustrates the characteristics of the above produced pea protein materials: pea protein example produced without enzymatic hydrolyzation (Example #1); a pea protein example produced with some enzymatic (protease) hydrolyzation (Example #2); and a pea protein example produced with some enzymatic (protein-glutaminase) hydrolyzation (Example #3).

TABLE 2
Pea Protein Material Examples: Evaluation Data

| Example No. | | Sensory Evaluation: Mouthfeel and Flavor |
|---|---|---|
| 1. | Non-Hydrolyzed | 1. Thickest, highest viscosity; some |
| | Pea Protein | pea/cooked vegetable flavor, no bitterness; |
| | Material (P870) | some slight gritty mouthfeel |
| 2. | Hydrolyzed | 2. Thinnest; very gritty mouthfeel; lots of |
| | Pea Protein | pea/cooked vegetable flavor, lots of |
| | Material (P870H) | bitter/metallic flavor |
| 3. | Enzyme Treated | 3. Middle thickness; creamy mouthfeel; |
| | Pea Protein | creamy appearance, no gritty mouthfeel; |
| | Material | milk flavor, very low pea/cooked vegetable |
| | (Experimental P2.0) | flavor; no bitter or metallic flavor |

Examples #1 P870 *; #2 P870H*; and #3 P2.0 (Experimental) were organoleptically evaluated at room temperature, dissolved in water, in 10% solution concentration. [* P870 and P870H were commercial products supplied by PURIS (Minneapolis, Minn., USA).] Table 2 shows that enzyme hydrolyzation affected the perceived grainy mouthfeel and creamy mouthfeel of the pea protein material Example #2 (P870H). The enzyme treatment used to produce the non-soy, legume, protein material Example #3 (P2.0) did not create a grainy mouthfeel and did create a creamy mouthfeel. All three Examples were made with field peas.

Solubility Testing using Centrifuge (Test A)

TABLE 3
Amount of sedimentation after centrifugation
of several pea proteins

| | | Sediment | Sediment | Sediment |
|---|---|---|---|---|
| Example | | Buildup (mL) | Buildup (mL) | Buildup Avg. |
| Name | pH | Test Value | Test Value | (mL) Test Value |
| Competitor | 6.62 | 23 | 22 | 22.5 |
| P870 | 6.78 | 20 | 18 | 19 |
| P870H | 6.71 | 12 | 14 | 13.0 |
| B2122 | 6.85 | 5 | 4 | 4.5 |
| B1140 | 6.81 | 4 | 4 | 4 |

Test Method:
1. Made a 10% solution of selected protein example in water at 70 F.
2. Mixed protein and water together for 10 minutes.
3. Recorded pH.
4. Then, filled test tube to 45 ml and ran the example in a centrifuge at 3500 RPM for 3 minutes. Each example was run in duplicates.
5. Reported amount of sediment present in each tube and averaged results across runs.

### Examples

(#1) Competitor=Bob Red Mills Unsweetened/Unflavored Pea Protein Powder
(#2) P870=PURIS Pea Protein 870
(#3) P870H=PURIS Pea Protein 870H
(#4) B2122=P2.0 122F=PURIS Pea Protein 2.0 Trial 1 processed at 50 C. [Enzyme hydrolysis done at 50 C.]
(#5) B1140=P2.0 140F=PURIS Pea Protein 2.0 Trial 2 processed at 60 C. [Enzyme hydrolysis done at 60 C.]

Conclusion: Based on the results presented in Table 3, it can be seen that examples B2122 (Protein 2.0 processed at 50 C 2^{nd} pilot trial) and B1140 (Protein 2.0 processed at 60 C 2^{nd} pilot trial) showed significant reduction in sediment buildup compared to the other examples. This agrees with theoretical thinking that deamination of glutamate by added protein-glutaminase enzyme at least in part changed the tertiary structure of the proteins, which allowed for greater interaction with water, and thus improved solubility and reduced sedimentation. This also illustrated a range of temperatures (e.g., 50-60 C) could be used to create the disclosed non-soy, legume, protein material with good solubility and reduced sedimentation.

Nitrogen Solubility Index (NSI) (Test B)

TABLE 4
Nitrogen Solubility Index Results

| Example: | Example Description | NSI Test Value |
|---|---|---|
| #1 | Competitor | 19.90% |
| #2 | P870 | 29.57% |
| #3 | P870H | 32.48% |
| #4 | P 2.0 Batch 1 - 50 C. Process | 58.68% |
| #5 | P2.0 Batch 2 - 60 C. Process | 96.50% |

Test Method: Nitrogen Solubility Index (NSI) [American Oil Chemist's Society (AOCS) Method Ba 11-65]
1. Weighed 20±0.1 example.
2. Filled 300 ml volumetric flask with distilled water at 25±1 C.
3. Poured 50 ml of the water into a blender cup.
4. Transferred the weighed example quantitatively to the blender cup. Stirred with a spatula to form a paste. Added remainder of the water to rinse the spatula and blender cup walls. Placed cup in position for blending.
5. Blended the example for 20 min at 120 rpm.
6. Removed the blender cup and poured the slurry into a 600 ml beaker. After the slurry had been separated, decanted, or pipetted a portion of the upper layer into a 50 ml centrifuge tube for 10 min at 2700 RPM.
7. Pipetted 15 ml of supernatant liquid into a Kjeldahl flask and determined the Nitrogen value.
8. NSI=% water dispersible protein/total protein×100.

### Examples

(#1) Competitor=Bob Red Mills Unsweetened/Unflavored Pea Protein Powder
(#2) P870=PURIS Pea Protein 870
(#3) P870H=PURIS Pea Protein 870H
(#4) P2.0 122F=PURIS Pea Protein 2.0 Trial 1 processed at 50 C. [Enzyme hydrolysis done at 50 C.]
(#5) P2.0 140F=PURIS Pea Protein 2.0 Trial 2 processed at 60 C. [Enzyme hydrolysis done at 60 C.]

Conclusion: Based on the results presented in Table 4, it can be seen that Example #4 (Protein 2.0 processed at 50 C 2^{nd} pilot trial) and Example #5 (Protein 2.0 processed at 60 C 2^{nd} pilot trial) showed at least in part an increase in solubility compared to the other examples. This agrees with theoretical thinking that deamination of glutamate by the added enzyme at least in part changed the tertiary structure of the proteins, which allowed for greater interaction with water, and thus greater solubility. This also illustrates that a range of enzyme hydrolysis procedure temperatures (e.g., 50-60 C) can be used to create the disclosed non-soy, legume, protein material with improved solubility.

Protein Dispersibility Index (PDI) (Test C)

TABLE 5
Protein Dispersibility Index Results

| Example | Example Description | PDI Test Value |
|---|---|---|
| #1 | Competitor | 14.52% |
| #2 | P870 | 88.50% |
| #3 | P870H | 54.50% |
| #4 | P 2.0 Batch 1 - 50 C. Process | 95.30% |

Test Method: Protein Dispersibility Index (PDI) [American Oil Chemist's Society (AOCS) Method Ba 10-65]
1. Weighed 20±0.1 example.
2. Filled 300 ml volumetric flask with distilled water at 25±1 C.
3. Poured 50 ml of the water into a blender cup.
4. Transferred the weighed sample quantitatively to the blender cup. Stirred with a spatula to form a paste. Added remainder of the water to rinse the spatula and blender cup walls. Placed cup in position for blending.
5. Blended the example for 20 min at 8500 rpm.
6. Removed the blender cup and poured the slurry into a 600 ml beaker. After the slurry had been separated, decanted, or pipetted a portion of the upper layer into a 50 ml centrifuge tube for 10 min at 2700 RPM.
7. Pipetted 15 ml of supernatant liquid into a Kjeldahl flask and determined the Nitrogen value.
8. PDI=% water dispersible protein/total protein×100.

### Examples

(#1) Competitor=Bob Red Mills Unsweetened/Unflavored Pea Protein Powder
(#2) P870=PURIS Pea Protein 870
(#3) P870H=PURIS Pea Protein 870H
(#4) P2.0 122F=PURIS Pea Protein 2.0 Trial 1 processed at 50 C. [Enzyme hydrolysis done at 50 C.]

Conclusion: Based on the results presented in Table 5, it can be seen that Example #4 (Protein 2.0 processed at 50 C 2^{nd} pilot trial) showed significant increase in dispersibility compared to the other examples. This agrees with theoretical thinking that deamination of glutamate by the added enzyme at least in part changed the tertiary structure of the proteins, which allowed for greater interaction with water, and thus greater solubility. The PDI testing method employs a more aggressive mixing step than the NSI test method, which would at least partially explain the differences in protein solubility results between the two methods.

### Sensory Test (Test D)

TABLE 6
Sensory Test Results: Bitterness, Saltiness, Cooked Pea/Vegetable Notes

| | P2.0 50 C. | | P2.0 60 C. | | P870 | | P870H | | Competitor | | China | |
|---|---|---|---|---|---|---|---|---|---|---|---|---|
| | Average | | Average | | Average | | Average | | Average | | Average | |
| | Test | | Test | | Test | | Test | | Test | | Test | |
| | Value | SD | Value | SD | Value | SD | Value | SD | Value | SD | Value | SD |
| Bitterness | 3.25 | 1.75 | 3.26 | 2.17 | 4.18 | 2.39 | 4.65 | 2.55 | 4.93 | 3.04 | 4.25 | 1.99 |
| Saltiness | 3.13 | 1.17 | 2.91 | 1.23 | 3.68 | 2.05 | 4.02 | 2.64 | 3.11 | 1.51 | 3.88 | 1.50 |
| Cooked | 3.76 | 2.19 | 2.55 | 1.26 | 3.16 | 1.34 | 4.25 | 2.09 | 4.72 | 3.52 | 4.77 | 1.46 |
| Pea/Vegetable | | | | | | | | | | | | |
| Notes | | | | | | | | | | | | |

Panelist test value averages and standard deviations for flavor attributes.

### Examples

P2.0 122F=PURIS Pea Protein 2.0 Trial 1 processed at 50 C. [Enzyme hydrolysis done at 50 C.]
P2.0 140F=PURIS Pea Protein 2.0 Trial 2 processed at 60 C. [Enzyme hydrolysis done at 60 C.]
P870=PURIS Pea Protein 870
P870H=PURIS Pea Protein 870H
Competitor=Bob Red Mills Unsweetened/Unflavored Pea Protein Powder
China=Yantai Oriental Protein Tech Pea Protein 80%

Sensory Test Method (Trained Panelists; n=7): Panelists were trained using specific materials (listed below) for each flavor attribute (i.e., bitterness, saltiness, cooked pea/vegetable notes) in the non-soy, legume, protein material examples. In these examples, the non-soy legume was made from yellow field peas. The examples were 10% solutions in water. The sensory test of the pea protein material examples was done blind, in random order, and using a 15 point scale (0=none or low; 15=significantly present).

Training Materials:
1. Bitterness:
   Caffeine solution (at 0.02%; 0.05%; 0.08%)
2. Saltiness:
   Sodium chloride solution (at 0.1%; 0.2%; 0.35%)
3. Cooked pea/vegetable notes:
   Cooked pea slurry (200 g peas/500 g water; 300 g peas/500 g water; 400 g peas/500 g water)

Sensory Anchors

| Attribute | Anchors (score on 15 point line) | | |
|---|---|---|---|
| Bitterness | Caffeine 0.02% | Caffeine 0.05% | Caffeine 0.08% |
| | (2.0) | (5.0) | (10.0) |
| Saltiness | Salt 0.1% | Salt 0.2% | Salt 0.35% |
| | (2.0) | (5.0) | (10.0) |
| Cooked | Pea Slurry | Pea Slurry | Pea Slurry |
| Pea/Vegetable | (2.0) | (7.0) | (12.0) |
| Notes | | | |

Conclusion:

The order of examples as to bitterness (highest to lowest): Competitor; P870H; China; P870; P 2.0 (60 C); and P 2.0 (50 C). Bitterness is a negative organoleptic trait. The protein material with lowest perceived amount of bitterness would be preferred by consumers. Product formulators would need to formulate to cover the bitterness. The results in Table 6 show that both P 2.0 examples had less bitter character than the other examples. The difference in processing enzyme hydrolyzation temperature did not cause obvious differences in bitterness level.

The order of examples as to saltiness (highest to lowest): P870H; China; P870; P 2.0 (50 C); Competitor; P 2.0 (60 C). Saltiness is a potentially negative organoleptic trait. The protein material with lowest perceived amount of saltiness might be preferred by consumers. Salt is known by product formulators to be a flavor enhancement tool. Its presence could cause the enhancement of both positive and negative sensory traits in any food product the protein is used. The difference in processing enzyme hydrolyzation temperature appeared to make a small difference in cooked pea/vegetable notes level.

The order of examples as to cooked pea/vegetable notes (highest to lowest): China; Competitor; P870H; P2.0 (50 C); P870; P 2.0 (60 C). The cooked pea/vegetable notes is a potentially negative organoleptic trait. The protein material with lowest perceived amount of cooked pea/vegetable notes might be preferred by consumers. Cooked pea/vegetable notes would be an organoleptic trait that product formulators would need to formulate around if used in mild flavored food products, such as dairy and dairy analog products. The difference in processing enzyme hydrolyzation temperature did appear to cause a difference in cooked pea/vegetable notes level, with P 2.0 60 C temperature having less cooked pea/vegetable notes than P 2.0 50 C. Both P 2.0 examples had less cooked pea/vegetable notes than P870H, Competitor, and China examples.

TABLE 7
Sensory Test Results: Texture (Viscosity, Amount of Particles, Creamy/Milky

| | P2.0 50 C. | | P2.0 60 C. | | P870 | | P870H | | Competitor | | China | |
|---|---|---|---|---|---|---|---|---|---|---|---|---|
| | Average | | Average | | Average | | Average | | Average | | Average | |
| | Test | | Test | | Test | | Test | | Test | | Test | |
| | Value | SD | Value | SD | Value | SD | Value | SD | Value | SD | Value | SD |
| Viscosity | 3.56 | 0.91 | 4.42 | 1.78 | 4.52 | 1.74 | 2.67 | 2.04 | 2.63 | 0.75 | 3.18 | 0.40 |
| Amount of | 2.33 | 1.41 | 1.98 | 1.80 | 2.57 | 1.61 | 6.5 | 2.93 | 6.2 | 2.62 | 5.81 | 1.43 |
| Particles | | | | | | | | | | | | |
| Creamy/Milky | 8.95 | 2.89 | 7.23 | 3.55 | 9.03 | 3.77 | 5.5 | 2.85 | 6.02 | 4.45 | 4.56 | 2.59 |
| Mouthfeel | | | | | | | | | | | | |

### Examples

P2.0 122F=PURIS Pea Protein 2.0 Trial 1 processed at 50 C
P2.0 140F=PURIS Pea Protein 2.0 Trial 2 processed at 60 C
P870=PURIS Pea Protein 870
P870H=PURIS Pea Protein 870H
Competitor=Bob Red Mills Unsweetened/Unflavored Pea Protein Powder
China=Yantai Oriental Protein Tech Pea Protein 80%

Sensory Test Method (Trained Panelists; n=7): Panelists were trained using specific materials (listed below) for each texture attribute (i.e., viscosity, amount of particles [grittiness], creamy/milky mouthfeel) in the non-soy, legume, protein material examples. In these examples, the non-soy legume was field peas. The examples were 10% solutions in water. The sensory test of the pea protein material examples was done blind, in random order, and using a 15 point scale (0=none or low; 15=significantly present).

Training Materials:
1. Viscosity:
   Water; Heavy Cream [Market Pantry Brand]; Sweetened condensed milk [Nestle Carnation Brand]
2. Amount of particles:
   30 g Chocolate putting [Hunts Snack Pack brand]+0.2 g PURIS RTE Pea Fiber [80 mesh];
   30 g Chocolate putting [Hunts Snack Pack brand]+1.0 g PURIS RTE Pea Fiber [80 mesh]; 30 g Chocolate putting [Hunts Snack Pack brand]+3.0 g PURIS RTE Pea Fiber [80 mesh]
3. Creamy/Milky Mouthfeel:
   Water; Skim milk [Kemps brand]; Half & Half [Land O' Lakes brand]; Heavy Cream [Market Pantry brand]

Sensory Anchors

| Attribute | Anchors (score on 15 point line) | | | |
|---|---|---|---|---|
| Viscosity | Water | Heavy Cream | Chocolate Syrup | Sweetened |
| | (1.0) | (4.0) | (9.0) | Condensed Milk |
| | | | | (14.5) |
| Amount of | Pudding + Fiber | Pudding + Fiber | Pudding + Fiber | |
| Particles | (2.5) | (6.0) | (12.0) | |
| Creamy/Milky | Water | Skim Milk | Half & Half | Heavy Cream |
| Mouthfeel | (0.0) | (3.0) | (8.0) | (14.0) |

Conclusion:

The order of examples as to viscosity (highest to lowest): P870; P 2.0 (60 C); P 2.0 (50 C); China; P 8709H; Competitor. Creating viscosity is a positive functional and organoleptic trait, though for some food products, too much thickness could limit the amount of protein material that could be added to a food product. The results in Table 7 show that both P2.0 examples (examples that are embodiments of the present disclosure) have greater viscosity than the other enzyme hydrolyzed protein example (P870H), the Competitor example, and the China example. So, less P2.0 would be required in a food product formulation to achieve a thicker end food product. The P2.0 examples having apparently less viscosity building property than P870, which means that more P2.0 could be added to a food product formulation than would be added with P870, in order to reach the same food product viscosity.

The order of examples as to amount of particles (highest to lowest): P870H; Competitor; China; P870; P2.0 (50 C); P2.0 (60 C). High amount of particles is a potentially negative organoleptic trait. Its presence would be a trait that product formulators would need to formulate around. The protein material with the lowest perceived amount of particles would be preferred by consumers. The difference in processing enzyme hydrolyzation temperature appeared to make a difference in amount of particles, with the higher temperature creating a lower amount of particles.

The order of examples as to creamy/milky mouthfeel (highest to lowest): P870, P2.0 (50 C); P2.0 (60 C); Competitor; P870H; China. The creamy/milky mouthfeel is a positive organoleptic trait. The protein material with the highest perceived amount of creamy/milky mouthfeel would be preferred by consumers. The creamy/milky mouthfeel organoleptic trait would be a trait that product developers could utilize in the formulation of beverages, and also in dairy and dairy analog products. The difference in processing enzyme hydrolyzation temperature did appear to cause a difference in cooked pea/vegetable notes level, with P2.0 (50 C) temperature having more creamy/milky mouthfeel than P2.0 (60 C). Both P2.0 examples had more creamy/milky mouthfeel than P870H, Competitor, and China examples.

### Particle Size Test

TABLE 8
Particle Size Distribution

| Pea Protein | | | | | | | |
|---|---|---|---|---|---|---|---|
| Type | <10% | <25% | <50% | <75% | <90% | <100% | >150% |
| P870 | 15.74μ | 23.20μ | 34.26μ | 48.60μ | 63.59μ | 309.6μ | 0.15μ |
| P870 H | 13.69μ | 21.00v | 31.36μ | 47.73μ | 68.18μ | 282.1μ | 0.32μ |
| P2.0 50 C. | 10.91μ | 16.67μ | 24.50μ | 34.68μ | 47.07μ | 111.10μ | 0μ |
| P2.0 60 C. | 10.51μ | 16.23μ | 24.28μ | 34.92μ | 47.81μ | 282.10μ | 0μ |
| Competitors | 47.53μ | 90.16μ | 145.90μ | 212.5μ | 287.80μ | 948μ | 48.20μ |

### Examples

P2.0 122F=PURIS Pea Protein 2.0 Trial 1 processed at 50 C
P2.0 140F=PURIS Pea Protein 2.0 Trial 2 processed at 60 C
P870=PURIS Pea Protein 870
P870H=PURIS Pea Protein 870H
Competitor=Bob Red Mills Unsweetened/Unflavored Pea Protein Powder

Test Method:

Used Beckman Coulter LS 1330 Particle Size Analyzer to measure particle size via Frauhofler Method (an IR Method). Representative samples of each example were measured for particle size distribution.

Results:

Results in Table 8 illustrate that examples Competitor and P870H have more of their particle of their size distribution shifted to larger particle size than the other Examples tested (comprising both of the P2.0 examples). This distribution shift puts more spray dried non-soy, legume, protein material particles in the size range that a tongue can feel, so that solutions of these non-soy, legume, protein material examples in water have a gritty, not creamy mouthfeel. The two P2.0 examples have particle distributions with less of their material being in this larger particle size range.

These results equate favorably with the Sensory Test results already discussed. That is, the two P2.0 examples had less of their particles in the size range that the tongue could perceive them as grit.

Conclusion:

Particle size of spray dried protein material effects the mouthfeel of the protein material in solution and in food products. As shown in Table 8, the Examples have different particle size profiles, especially at the larger particle sizes. Not to be bound by theory, the non-soy, legume, protein material of this disclosure (Examples P2.0 [50 C] and P2.0 [60 C]) had a smoother, creamier, less gritty (less amount of particles) than Competitor and P870H.

The parties of this disclosure do recognize that all of these examples were not spray dried on the same equipment. And it has already been discussed that several spray drying factors can effect particle character. But these particle size results are still useful in assisting in explaining why P 2.0 (50 C) and P 2.0 (60 C) embodiments of the present disclosure have mouthfeel texture characteristics different from that of the other Examples.

### Flowability Test

TABLE 9
Flowability Measurement using Consistometer
(Bostwick) Viscometer

| Example Description | Flowability (cm) Test Value |
|---|---|
| P870 | 0.0 |
| P870H | 23.5 |
| P2.0 60 C. | 7.6 |
| P2.0 50 C. | 1.5 |
| Competitor | 0.0 |

### Examples

P2.0 50 C=PURIS Pea Protein 2.0 Trial 1 processed at 50 C
P2.0 60 C=PURIS Pea Protein 2.0 Trial 2 processed at 60 C
P870=PURIS Pea Protein 870
P870H=PURIS Pea Protein 870H
Competitor=Bob Red Mills Unsweetened/Unflavored Pea Protein Powder

Test Method:
1. Created a 20% solids solution by mixing example protein material and water together. Water was at 21 C.
2. Once a homogenous mixture was formed, mixture was placed into the consistometer sample box.
3. Sample box lever was triggered and the distance the mixture flowed in 30 seconds was recorded.

Conclusion:

Based on the results presented in the following Table 9 a difference in flowability can be seen between each Example. P870H produced a mixture that is very flowable with a test value of 23.5 cm. Compared to the P870 mixture that did not flow under these conditions. As previously discussed, P870H is a pea protein material that has a protease enzyme treatment. Protein 2.0 processed at 60 C had some flowability with a test value of 7.6 cm, which makes Protein 2.0 a great protein material choice by a product formulator wanting to create high content protein products (e.g., beverages) without having the disadvantages of excess thickening in food products (such as a product formulator could get with high content levels of P870 and Bobs non-soy, legume, protein materials). P2.0 also had the advantage of being better tasting than the other Examples.

P2.0 (processed at 50 C) Example had a flowability test value of 1.5 compared to the P2.0 (processed at 40 C) Example test value of 7.6. Differences in flowability test values between the two P2.0 Examples test values could be due to the higher temperature processing time of P2.0 (60 C) giving the protein-glutaminase more energy to use in its hydrolysis of the pea protein in the pea non-soy, legume, protein material. The added energy, though, was not enough to create the gritty texture present in the other non-soy, legume, protein materials, as already discussed in this disclosure.

Overall, the non-soy, legume, protein material of this disclosure, illustrated using pea protein material that was produced by the method of this disclosure, had better functionality and better flavor than the comparison Examples (i.e., P870H, P870, Competitor, and China).

### Examples: Food Products with Non-Soy, Legume, Protein MATERIAL

TABLE 10
Pea Milk with P870 Formula

| INGREDIENT | |
|---|---|
| INFORMATION | FORMULATION |
| Ingredient Description | % WT |
| Water | 85-95% |
| PURIS Pea Protein P870 | 4-7% |
| Oil (e.g., Sunflower Oil High Oleic) | 2-5% |
| Salt | 0-0.15% |
| Hydrocolliod (e.g., Gellan Gum) | 0-0.10% |
| Sugar | 0.5-3% |
| Natural Flavors | 0-2.0% |
| TOTALS | 100.000% |

TABLE 11
Pea Milk with Protein 2.0 Formula

| INGREDIENT | |
|---|---|
| INFORMATION | FORMULATION |
| Ingredient Description | % WT |
| Water | 85-95% |
| PURIS Pea Protein 2.0 | 4-7% |
| Oil (e.g., Sunflower Oil High Oleic) | 2-5% |
| Salt | 0-0.15% |
| Hydrocolliod (e.g., Gellan Gum) | 0-0.10% |
| Sugar | 0.5-3% |
| Natural Flavors | 0-2.0% |
| TOTALS | 100.000% |

TABLE 12
Pea Milk with Protein 2.0 No Gellan Gum Formula

| INGREDIENT | |
|---|---|
| INFORMATION | FORMULATION |
| Ingredient Description | % WT |
| Water | 85-95% |
| PURIS Pea Protein 2.0 | 4-7% |
| Oil (e.g., Sunflower Oil High Oleic) | 2-5% |
| Salt | 0.0-0.15% |
| Natural Flavors | 0-2.0% |
| Sugar | 0.5-3% |
| TOTALS | 100.000% |

Method: Pea Milk Instructions
1. Using a high shear mixer:
   a. Mixed gum into the water until completely incorporated.
   b. Added stevia powder to the mixture.
   c. Added buffering salt to the mixture.
   d. Added PURIS Pea Protein, mixed well, and hydrated for about 5 minutes.
   e. Slowly added the sunflower oil and then mixed for several minutes.
   f. Lastly combined and added sugar, guar fiber, and cocoa (Chocolate beverage).
2. Ran through Microthermics unit and homogenizer.
   a. Ran UHT at 88 C preheat and 140 C final heat for 6 seconds (indirect steam injection) and homogenized at 2500 psi. Homogenized between the preheat and final heating steps. Final product exited at 24 C.

Conclusion:

The color was very similar between Pea Milk made with each Example (Pea Milk with P870 vs. Pea Milk with P2.0). The Pea Milk with P2.0 had a slightly creamier mouthfeel and more body in the mouth than the Pea Milk with P870. Neither Pea Milk had noticeable grit. The Pea Milk with P2.0 tasted cleaner with less beany/pea notes. It also had slightly less amount of drying or astringent effect in the mouth. The Pea Milk with P2.0 also performed well throughout shelf life without gums (that is, no separation or synerises).

TABLE 13
Vanilla RTD with P870MV Formula

| INGREDIENT | |
|---|---|
| INFORMATION | FORMULATION |
| Ingredient Description | % WT |
| Water | 85-95% |
| PURIS Pea Protein P870MV | 4-10% |
| Oil ( e.g., Sunflower Oil High Oleic) | 0-3% |
| Hydrocolloid (e.g., Guar Fiber) | 0-3% |
| Hydrocolloid (e.g., Gellan Gum) | 0-0.1% |
| Sugar | 0.5-3.0% |
| Natural Flavors | 0-2% |
| Dipotassium Phosphate | 0-1.0% |
| HIS (e.g., Stevia) | 0-0.1% |
| TOTALS | 100.000% |

Note:
P870MV is a product of PURIS that is between P870 and P870H.

TABLE 14
Vanilla RTD with Protein 2.0 Formula

| INGREDIENT | |
|---|---|
| INFORMATION | FORMULATION |
| Water | 85-95% |
| PURIS Pea Protein P2.0 | 4-10% |
| Oil ( e.g., Sunflower Oil High Oleic) | 0-3% |
| Hydrocolloid (e.g., Guar Fiber) | 0-3% |
| Hydrocolloid (e.g., Gellan Gum) | 0-0.1% |
| Sugar | 0.5-3.0% |
| Natural Flavors | 0-2% |
| Dipotassium Phosphate | 0-1.0% |
| HIS (e.g., Stevia) | 0-0.1% |
| TOTALS | 100.000% |

TABLE 15
Chocolate RTD with P870MV Formula

| INGREDIENT | |
|---|---|
| INFORMATION | FORMULATION |
| Water | 85-95% |
| PURIS Pea Protein P870MV | 4-10% |
| Oil ( e.g., Sunflower Oil High Oleic) | 0-3% |
| Hydrocolloid (e.g., Guar Fiber) | 0-3% |
| Hydrocolloid (e.g., Gellan Gum) | 0-0.1% |
| Sugar | 0.5-3.0% |
| Water | 85-95% |
| Cocoa Powder | 0-3% |
| Natural Flavors | 0-2% |
| Dipotassium Phosphate | 0-1.0% |
| HIS (e.g., Stevia) | 0-0.1% |
| TOTALS | 100.000% |

TABLE 16
Chocolate RTD with Protein 2.0 Formula

| INGREDIENT | |
|---|---|
| INFORMATION | FORMULATION |
| Ingredient Description | % WT |
| Water | 85-95% |
| PURIS Pea Protein P2.0 | 4-10% |
| Oil ( e.g., Sunflower Oil High Oleic) | 0-3% |
| Hydrocolloid (e.g., Guar Fiber) | 0-3% |
| Hydrocolloid (e.g., Gellan Gum) | 0-0.1% |
| Sugar | 0.5-3.0% |
| Water | 85-95% |
| Cocoa Powder | 0-3% |
| Natural Flavors | 0-2% |
| Dipotassium Phosphate | 0-1.0% |
| HIS (e.g., Stevia) | 0-0.1% |

Method: Ready-To-Drink (RTD) Instructions
1. Using a high shear mixer:
   a. Mixed gum into the water until completely incorporated.
   b. Added stevia powder to the mixture.
   c. Added buffering salt to the mixture.
   d. Added PURIS Pea Protein and hydrated for about 5 minutes.
   e. Slowly added the sunflower oil and let mix for several minutes.
   f. Lastly combined and added sugar, guar fiber, and cocoa (Chocolate beverage).
2. Ran through Microthermics unit & homogenizer.
   a. Ran UHT at 88 C preheat and 141 C final heat for 6 seconds (indirect steam injection) and homogenized at 2500 psi. Homogenized between the preheat and final heating step. Final Product exited at 24 C.

Results:

RTD-P870MV: The flavor of the RTD made with P870MV was less creamy and more beany and plant flavored than the RTD with Protein 2.0 (P2.0). The RTD with P870MV was also thicker and had a more gritty texture than the RTD made with P2.0. The RTD with P870MV was slightly more white/tan than the RTD with P2.0.

RTD-P2.0: The flavor of the RTD with P2.0 had more vanilla flavor and less or no beany flavor notes. The RTD with P2.0 was slightly more yellow than the RTD made with P870MV. The RTD with P2.0 had a smoother, more creamy, no grittiness texture than the RTD with P870MV.

Conclusion:

RTD with Protein 2.0 was found to have an acceptable mouthfeel and overall flavor profile. RTD made with Protein 2.0 imparted more vanilla and/or chocolate aroma and flavor when compared to RTD made with P870MV. RTD with Protein 2.0 was also slightly thinner and had a smoother mouthfeel when compared to RTD made with P870MV. When P870 was used, the RTD had significant gelling problems during shelf life and would have flavor issues due to high protein addition percent usages.

By using Protein 2.0 (the product of the present disclosure), product formulators will be able to effectively move past the 20 g (per 100 g serving) of plant protein per bottle addition limit that most beverages stop at. Formulators, using Protein 2.0, will be able to provide beverages with at least 30 g (per 100 g serving) of plant protein per bottle.

TABLE 17
Cream Cheese with P870 Formula

| INGREDIENT | |
|---|---|
| INFORMATION | FORMULATION |
| Ingredient Description | % WT |
| Water | 50-75% |
| PURIS Pea Protein P870 | 1-8% |
| Oil (e.g., Coconut) | 18-35% |
| Sugar (e.g. Dextrose) | 0-8% |
| PURIS Pea Starch (Native) | 0-6% |
| Salt | 0-3% |
| TOTALS | 100.000% |

TABLE 18
Cream Cheese with Protein 2.0 Formula

| INGREDIENT | |
|---|---|
| INFORMATION | FORMULATION |
| Ingredient Description | % WT |
| Water | 50-75% |
| PURIS Pea Protein P2.0 | 1-8% |
| Oil (e.g., Coconut) | 18-35% |
| Sugar (e.g. Dextrose) | 0-8% |
| PURIS Pea Starch (Native) | 0-6% |
| Salt | 0-3% |
| TOTALS | 100.000% |

Method: Cream Cheese Instructions
1. Mixed ingredients using high shear mixer at 10,000-12,000 rpm.
2. Transferred cream cheese batter into Thermomix and pasteurized product to 93 C. Took approximately 10-15 minutes to meet temperature requirements.
3. Transferred product to homogenizer and homogenized at 2500-3000 psi (2 stage 2000, 500 psi).
4. Cooled product. Added non-dairy cultures (Vivopel MSM 981) and placed in incubator at 25 C-26 C.
5. Added citric acid (acidulant) to lower pH from 4.8 to 4.2.
6. Cut product using a hand mixer.

Conclusion:

Cream Cheese with Protein 2.0 was found to have acceptable viscosity and mouthfeel, Cream Cheese with Protein 2.0 was found to have a more creamy mouthfeel than the Cream Cheese with P870. Cream Cheese with Protein 2.0 was found to have acceptable flavor—that is, without bitterness or appreciable pea/cooked vegetable flavor.

TABLE 19
Yogurt with P870MV & P870 Formula

| INGREDIENT | |
|---|---|
| INFORMATION | FORMULATION |
| Ingredient Description | % WT |
| Water | 70-88% |
| PURIS Pea Protein 870MV/870 (90/10) | 3-10% |
| Oil (e.g., Coconut Oil) | 0.5-6% |
| Sugar (e.g., Sucrose) | 1-6% |
| PURIS Pea Starch (Native) | 0-6% |
| TOTALS | 100.000% |

Note: Formula uses 90/10% blend of P870MV and P870 because each alone would result in an unacceptable product viscosity for processing and consumption.

TABLE 20
Yogurt with Protein 2.0 Formula

| INGREDIENT | |
|---|---|
| INFORMATION | FORMULATION |
| Ingredient Description | % WT |
| Water | 70-88% |
| PURIS Pea Protein 2.0 | 3-10% |
| Oil (e.g., Coconut Oil) | 0.5-6% |
| Sugar (e.g., Sucrose) | 1-6% |
| PURIS Pea Starch (Native) | 0-6% |
| TOTALS | 100.000% |

Method: Yogurt Instructions
1. Mixed ingredients using a high shear mixer at 10,000-12,000 rpm.
2. Ran the base on a microthermix unit; preheated to 60 C.
3. Homogenized in two stage homogenizer (at 2000, 500 psi).
4. Pasteurized at 85 C for 30 seconds.
5. Product left the pasteurizer unit at 15 C-32 C.
6. Reheated product using a double boiler to a temperature of 43 C.
7. Added culture (Vivolac ABY 421 ND) to product per manufacturer's instructions and placed product in an incubator for 8 hours.
8. Cut product using a hand mixer.

Conclusion: Yogurt with Protein 2.0 was found to have an acceptable final viscosity and mouthfeel. Yogurt made with Protein 2.0 imparted more of a velvety mouthfeel while maintaining a thick texture that had a favorable cutable texture. Yogurt with Protein 2.0 was also milder in flavor and had slightly less noticeable astringency and beany notes when compared to yogurt made with P870. Overall, these benefits will help product formulators provide products with increased protein content, while also reducing the amount of flavors (e.g., flavor maskers) in their formulas.

Dry Beverage Blends (DBB) (Reconstituted by Consumer)

TABLE 21
Vanilla DBB with P870 Formula

| INGREDIENT | |
|---|---|
| INFORMATION | FORMULATION |
| Ingredient Description | % WT |
| Pea Protein 870 | 88-98% |
| Stevia | 0-0.6% |
| Monk Fruit Extract | 0-0.6% |
| Guar Gum | 0.2-0.9% |
| Natural Type Flavors | 0.0-2% |
| TOTALS | 100.000% |

TABLE 22
Vanilla DBB with P2.0 Formula

| INGREDIENT | |
|---|---|
| INFORMATION | FORMULATION |
| Ingredient Description | % WT |
| Pea Protein 2.0 | 88-98% |
| Stevia | 0-0.6% |
| Monk Fruit Extract | 0-0.6% |
| Guar Gum | 0.2-0.9% |
| Natural Type Flavors | 0-2% |
| TOTALS | 100.000% |

Method: Dry Beverage Blend Instructions
1. Dry blend all materials together.
2. Package.

Conclusion: Differences were noted between the Dry Beverage Blends (DBBs) made with P2.0 versus the DBBs made with P870, in particular DBB with P2.0 had a more creamy taste and mouthfeel compared to DBB made with P870. Also, the parties of this disclosure found that at least a 10% reduction in flavor and sweetener ingredients could be used in a DBB made with P2.0 and still have the same sweetness and flavor perception as a DBB with P870 and full ingredient level addition. This was due to the P2.0 non-soy, legume, protein material (made with peas) having an overall cleaner, milk-like taste that required less flavor and sweetener addition and less flavor masking than DBB made with P870.

Overall, the non-soy, legume, protein material of this disclosure (illustrated in this disclosure with the non-soy legume being field peas), which was produced by the method of this disclosure, performed better (that is, had greater and more favorable functionality and favor) than did the more hydrolyzed and the non-hydrolyzed pea protein examples.

The compositions and methods of the present disclosure are capable of being incorporated in the form of a variety of embodiments, only a few of which have been illustrated and described. The disclosure may be embodied in other forms without departing from its spirit or essential characteristics. The described embodiments are to be considered in all respects only as illustrative and not restrictive, and the scope of the disclosure, therefore, is indicated by the appended claims rather than by the foregoing description. All changes which come within the meaning and range of equivalency of the claims are to be embraced within their scope.

In sum, it is important to recognize that this disclosure has been written as a thorough teaching rather than as a narrow dictate or disclaimer. Reference throughout this specification to “one embodiment”, “an embodiment”, or “a specific embodiment” means that a particular feature, structure, or characteristic described in connection with the embodiment is comprised in at least one embodiment and not necessarily in all embodiments. Thus, respective appearances of the phrases “in one embodiment”, “in an embodiment”, or “in a specific embodiment” in various places throughout this specification are not necessarily referring to the same embodiment. Furthermore, the particular features, structures, or characteristics of any specific embodiment may be combined in any suitable manner with one or more other embodiments. It is to be understood that other variations and modifications of the embodiments described and illustrated herein are possible in light of the teachings herein and are to be considered as part of the spirit and scope of the present subject matter.

It will also be appreciated that one or more of the elements depicted in the drawings/figures can also be implemented in a more separated or integrated manner, or even removed or rendered as inoperable in certain cases, as is useful in accordance with a particular application. Additionally, any signal arrows in the drawings/Figures should be considered only as exemplary, and not limiting, unless otherwise specifically noted. Furthermore, the term “or” as used herein is generally intended to mean “and/or” unless otherwise indicated. Combinations of components or steps will also be considered as being noted, where terminology is foreseen as rendering the ability to separate or combine is unclear.

As used in the description herein and throughout the claims that follow, “a”, “an”, and “the” comprises plural references unless the context clearly dictates otherwise. Also, as used in the description herein and throughout the claims that follow, the meaning of “in” comprises “in” and “on” unless the context clearly dictates otherwise. Variation from amounts specified in this teaching can be “about” or “substantially,” so as to accommodate tolerance for such as acceptable manufacturing tolerances.

The foregoing description of illustrated embodiments, including what is described in the Abstract and the Modes, and all disclosure and the implicated industrial applicability, are not intended to be exhaustive or to limit the subject matter to the precise forms disclosed herein. While specific embodiments of, and examples for, the subject matter are described herein for teaching-by-illustration purposes only, various equivalent modifications are possible within the spirit and scope of the present subject matter, as those skilled in the relevant art will recognize and appreciate. As indicated, these modifications may be made in light of the foregoing description of illustrated embodiments and are to be included, again, within the true spirit and scope of the subject matter disclosed herein.

The resultant non-soy, legume, protein material can also be used to make supplements, pharmaceuticals, and industrial products. All mentions of the disclosed non-soy, legume, protein material towards use in food products, also implies similar use in supplements, pharmaceuticals and industrial products.

The compositions, articles, apparatuses, and methods of the present disclosure are capable of being incorporated in the form of a variety of embodiments, only a few of which have been illustrated and described. The disclosure may be embodied in other forms without departing from its spirit or essential characteristics. The described embodiments are to be considered in all respects only as illustrative and not restrictive, and the scope of the disclosure, therefore, is indicated by the appended claims rather than by the foregoing description. All changes which come within the meaning and range of equivalency of the claims are to be embraced within their scope.

Thus, although the foregoing disclosure has been described in some detail by way of illustration and example for purposes of clarity and understanding, it will be obvious that certain changes and modifications such as process modifications, formula adjustments and the like may be practiced within the scope of the disclosure, as limited only by the scope of the claims.

What is claimed is:

1. A non-soy, legume, protein material comprising:
a) at least 50% dry weight protein;
b) at least 20% of the dry weight protein is soluble at about 21 C at pH 4-8; and
c) wherein the non-soy, legume, protein material has a sedimentation level test value of less than about 10 as measured by Solubility Testing Using Centrifuge (Test A).

2. The non-soy, legume, protein material of claim 1, wherein the non-soy, legume, protein material has a Nitrogen Solubility Index test value of greater than about 40% as measured by Nitrogen Solubility Index (Test B).

3. The non-soy, legume, protein material of claim 1, wherein the non-soy, legume protein material has a Protein Dispersibility Index test value of greater than about 70% as measure by Protein Dispersibility Index (Test C).

4. The non-soy, legume, protein material of claim 1, wherein the non-soy, legume protein material has a Sensory Test test value of less than 4 in bitterness, saltiness, and cooked pea flavor notes as measured by Sensory Test (Test D).

5. The non-soy, legume, protein material of claim 1, wherein the non-soy, legume protein material has a Sensory Test test value of greater than 3 in mouthfeel viscosity and a Sensory Test test value of greater than 7 in mouthfeel creaminess as measured by Sensory Test (Test D).

6. The non-soy, legume, protein material of claim 1, wherein the non-soy legume protein material comprises at least 20% pea protein.

7. A process of making a non-soy, legume, protein material of claim 1, wherein the process comprises the steps of:
a) grinding de-hulled non-soy legumes to make a ground non-soy legume matter;
b) mixing the ground non-soy legume matter with water to make an intermediate slurry;
c) separating insoluble fiber and starch portions from a soluble protein portion of the intermediate slurry to make an intermediate protein portion slurry;
d) coagulating protein in the intermediate protein portion slurry to make a coagulated protein;
e) removing the coagulated protein from the intermediate protein portion slurry and
solubilizing the coagulated protein in water;
f) neutralizing the coagulated protein solubilized in water to make a neutralized protein slurry;
g) intermixing the neutralized protein slurry with enzyme material;
h) heating the neutralized protein slurry containing enzyme to about 32 C-121 C to make a heated neutralized protein slurry; and
i) removing water from the heated neutralized protein slurry to make a non-soy, legume, protein material.

8. The process of claim 7, wherein the enzyme material used is a deaminating enzyme.

9. The process of claim 7, wherein the enzyme material used is a bacterial strain of Chryseobacterium proteolyticum.

10. The process of claim 7, further comprising the step of heating of the neutralized protein slurry containing enzyme to a temperature between 32 C-65 C.

11. The process of claim 7, further comprising the step of heating the neutralized protein slurry containing enzyme for 5 minutes to 6 hours.

12. The process of claim 7, further comprising the step of heating of the neutralized protein slurry containing enzyme in at least two heating processes, of which one heating processes is performed at least at a temperature of 93 C.

13. The non-soy, legume, protein material of claim 1, wherein at least 70% by dry weight of the dry weight protein is in globular form and at least 5% by dry weight of the dry weight protein is in albumin form.

14. The non-soy, legume, protein material of claim 14, wherein the non-soy, legume, protein material has a PDCAAS of 0.75-1.00.

15. The non-soy, legume, protein material of claim 1, wherein at least 65% by dry weight of the dry weight protein is from non-soy legumes, and at least 5% by dry weight of the dry weight protein is from nuts, grains, vegetables, fruits, or combinations of such.

16. The non-soy, legume, protein material of claim 16, wherein the non-soy, legume, protein material has a PDCAAS of 0.75-1.00.

17. The process of claim 7, wherein the enzyme material comprises both a protease enzyme and a deaminating enzyme.

18. A food product containing the non-soy, legume, protein material of claim 1, wherein the food product is selected from a group consisting essentially of beverages, sauces, soups, meat analogs, egg analogs, non-dairy alternatives, cheese analogs, extruded products, powders, mixes, bakery products, and combinations thereof.

19. A product containing the non-soy, legume, protein material of claim 1, wherein the product is selected from a group consisting essentially of human food, animal food, supplements, pharmaceuticals, industrial products, and combinations thereof.
